(19) United States
(12) Patent Application Publication
(10) Pub. No.: US 2024/0383975 A1
(43) Pub. Date: Nov. 21, 2024

(54) COMPOSITION FOR TREATING OR PREVENTING IMMUNOLOGICAL ABNORMAL INFLAMMATORY DISEASES

(71) Applicants: Kazuyuki Yoshizaki, Kobe-shi (JP); Yoshikane Kikushige, Fukuoka-shi (JP); Hiroaki Niiro, Fukuoka-shi (JP); Takuya Harada, Fukuoka-shi (JP); Koichi Akashi, Fukuoka-shi (JP)

(72) Inventors: Kazuyuki Yoshizaki, Kobe-shi (JP); Yoshikane Kikushige, Fukuoka-shi (JP); Hiroaki Niiro, Fukuoka-shi (JP); Takuya Harada, Fukuoka-shi (JP); Koichi Akashi, Fukuoka-shi (JP)

(21) Appl. No.: 18/692,254

(22) PCT Filed: Jul. 29, 2022
(86) PCT No.: PCT/JP2022/029363

§ 371 Date: Mar. 14, 2024

(30) Foreign Application Priority Data
Sep. 15, 2021 (WO) PCT/JP2021/033984

(51) Int. Cl. C07K 16/24; A01K 67/0271; A61K 45/06; A61P 29/00

(52) U.S. Cl. CPC C07K 16/24; A01K 67/0271; A61K 45/06; A61P 29/00; A01K 2227/105; A01K 2267/0368; A01K 2267/0387; C07K 2317/24

(57) ABSTRACT

The present disclosure provides an excellent pharmaceutical composition for treatment or prevention of an immune-abnormal inflammatory disease. According to the present disclosure, there is provided a composition for preventing or treating an immune-abnormal inflammatory disease, comprising a modulator of CXCL13, CXCR5, and/or peripheral helper T cells (Tph cells) [CXCL13/CXCR5/Tph]. According to the present disclosure, a model animal of an immune-abnormal inflammatory disease represented by Castleman disease and the like and a method for producing the same are also provided.

### TECHNICAL FIELD

The present disclosure relates to compositions, methods, and the like for treating or preventing immune-abnormal inflammatory diseases. More specifically, the present disclosure relates to compositions, methods, and the like for treating or preventing diseases of lymphoproliferative and/or immune-abnormal diseases, such as Castleman disease.

### BACKGROUND ART

Castleman disease is a lymphoproliferative disorder characterized by the presence of giant lymph node proliferation pathologically accompanied by plasma cell infiltration. Patients with Castleman disease generally have fever, anemia, hypergammaglobulinemia, and elevated serum levels of acute-phase reactant proteins, all due to large amounts of IL-6 produced in the lymph nodes.

### SUMMARY OF THE INVENTION

### Problems to be Solved by the Invention

Among Castleman disease, idiopathic multicentric Castleman disease is a refractory lymphoproliferative disease. As a therapy, there is an IL-6 inhibitor, but it has been found that a sufficient effect is not obtained in about 20% of cases. Since it is a rare disease and there is no experimental animal model, development of a novel therapy has not progressed at present.

Accordingly, the present disclosure provides the following items.

### (Item 1)

A composition for preventing or treating an immune-abnormal inflammatory disease, comprising a modulator of CXCL13, CXCR5, and/or peripheral helper T cells (Tph cells) [CXCL13/CXCR5/Tph].

### (Item 2)

The composition according to the preceding item, wherein the modulator is selected from the group consisting of proteins, polypeptides, oligopeptides, peptides, polynucleotides, oligonucleotides, nucleotides, nucleic acids (including, for example, cDNA, DNA such as genomic DNA, and RNA such as mRNA), polysaccharides, oligosaccharides, lipids, organic small molecules (e.g., hormones, ligands, information transmitters, organic small molecules, molecules synthesized by combinatorial chemistry, small molecules that can be utilized as pharmaceuticals (e.g., small molecule ligands and the like), and the like), and complex molecules thereof.

### (Item 3)

The composition according to any one of the preceding items, wherein the modulator is:
an anti-CXCL13 antibody, a nucleic acid medicine for CXCL13, or a CXCL13 inhibitor or peptide,
an anti-CXCR5 antibody, a nucleic acid medicine for CXCR5, or a CXCR5 inhibitor or peptide, or
an anti-Tph cell antibody, a nucleic acid medicine for Tph cell, or a Tph cell inhibitor or peptide.

### (Item 4)

The composition according to any one of the preceding items, wherein the modulator is a modulator of CXCL13-CXCR5 interaction.

### (Item 5)

The composition according to any one of the preceding items, wherein the modulator is a modulator of Tph cells.

### (Item 6)

The composition according to any one of the preceding items, wherein the anti-CXCL13 antibody is a neutralizing antibody against CXCL13.

### (Item 7)

The composition according to any one of the preceding items, wherein the anti-CXCL13 antibody is a chimeric antibody, humanized antibody, or human antibody against CXCL13.

### (Item 8)

The composition according to any one of the preceding items, wherein the disease is a CXCL13-mediated disease.

### (Item 9)

The composition according to any one of the preceding items, wherein the disease comprises Castleman disease, rheumatoid arthritis, plasmacytosis, hyperimmunoglobulinemia, anemia, nephritis, cachexia, multiple myeloma, mesangial proliferative nephritis, systemic lupus erythematosus, systemic scleroderma, Crohn's disease, ulcerative colitis, pancreatitis, psoriasis, juvenile chronic arthritis, or systemic juvenile idiopathic arthritis, vasculitis, Kawasaki disease, TAFRO syndrome, and Poems syndrome.

### (Item 10)

The composition according to any one of the preceding items, wherein the disease comprises idiopathic multicentric Castleman disease.

### (Item A1)

An immune-abnormal inflammatory disease model animal.

### (Item A2)

The model animal according to the preceding item, wherein the immune-abnormal inflammatory disease is Castleman disease.

### (Item A3)

The model animal according to any one of the preceding items, wherein the immune-abnormal inflammatory disease is idiopathic multicentric Castleman disease.

### (Item A4)

The model animal according to any one of the preceding items, wherein the animal is an immunodeficient animal, and the immunodeficient animal contains lymph node cells derived from other than the immunodeficient animal.

### (Item A5)

The model animal according to any one of the preceding items, wherein the lymph node cells are lymph node cells of a patient with iMCD-NOS.

### (Item B1)

A method for producing an immune-abnormal inflammatory disease model animal, the method comprising a step of xenotransplanting into a non-human animal a graft derived from a patient suffering from an immune-abnormal inflammatory disease.

### (Item B2)

The method according to the preceding item, wherein the non-human animal comprises an immunodeficient animal.

### (Item B3)

The method according to any one of the preceding items, wherein the non-human animal comprises an immunodeficient mouse.

### (Item B4)

The method according to any one of the preceding items, wherein the graft comprises lymph node cells.

### (Item B5)

The method according to any one of the above items, wherein the lymph node cells are lymph node cells of a patient with iMCD-NOS.

### (Item B6)

The method according to any of the preceding items, wherein the disease comprises Castleman disease, rheumatoid arthritis, plasmacytosis, hyperimmunoglobulinemia, anemia, nephritis, cachexia, multiple myeloma, mesangial proliferative nephritis, systemic lupus erythematosus, systemic scleroderma, Crohn's disease, ulcerative colitis, pancreatitis, psoriasis, juvenile chronic arthritis, or systemic juvenile idiopathic arthritis, vasculitis, Kawasaki disease, TAFRO syndrome, and Poems syndrome.

### (Item B7)

The method according to any of the preceding items, wherein the disease comprises idiopathic multicentric Castleman disease.

### (Item B8)

A model animal produced by the method according to any one of the preceding items.

### (Item C1)

A method for preventing or treating an immune-abnormal inflammatory disease in a subject in need thereof, comprising administering to the subject an effective amount of a modulator of CXCL13, CXCR5, and/or peripheral helper T cells (Tph cells) [CXCL13/CXCR5/Tph].

### (Item C2)

The method according to the preceding item, wherein the modulator is selected from the group consisting of proteins, polypeptides, oligopeptides, peptides, polynucleotides, oligonucleotides, nucleotides, nucleic acids (including, for example, cDNA, DNA such as genomic DNA, and RNA such as mRNA), polysaccharides, oligosaccharides, lipids, organic small molecules (e.g., hormones, ligands, information transmitters, organic small molecules, molecules synthesized by combinatorial chemistry, small molecules that can be utilized as pharmaceuticals (e.g., small molecule ligands and the like), and the like), and complex molecules thereof.

### (Item C3)

The method according to any one of the preceding items, wherein the modulator is:
an anti-CXCL13 antibody, a nucleic acid medicine for CXCL13, or a CXCL13 inhibitor or peptide,
an anti-CXCR5 antibody, a nucleic acid medicine for CXCR5, or a CXCR5 inhibitor or peptide, or
an anti-Tph cell antibody, a nucleic acid medicine for Tph cell, or a Tph cell inhibitor or peptide.

### (Item C4)

The method according to any one of the preceding items, wherein the modulator is a modulator of CXCL13-CXCR5 interaction.

### (Item C5)

The method according to any one of the preceding items, wherein the modulator is a modulator of Tph cells.

### (Item C6)

The method according to any one of the preceding items, wherein the anti-CXCL13 antibody is a neutralizing antibody against CXCL13.

### (Item C7)

The method according to any one of the preceding items, wherein the anti-CXCL13 antibody is a chimeric antibody, humanized antibody, or human antibody against CXCL13.

### (Item C8)

The method according to any one of the preceding items, wherein the disease is a CXCL13-mediated disease.

### (Item C9)

The method according to any of the preceding items, wherein the disease comprises Castleman disease, rheumatoid arthritis, plasmacytosis, hyperimmunoglobulinemia, anemia, nephritis, cachexia, multiple myeloma, mesangial proliferative nephritis, systemic lupus erythematosus, systemic scleroderma, Crohn's disease, ulcerative colitis, pancreatitis, psoriasis, juvenile chronic arthritis, or systemic juvenile idiopathic arthritis, vasculitis, Kawasaki disease, TAFRO syndrome, and Poems syndrome.

### (Item C10)

The method according to any of the preceding items, wherein the disease comprises idiopathic multicentric Castleman disease.

### (Item D1)

Use of a modulator of CXCL13, CXCR5, and/or peripheral helper T cells (Tph cells) [CXCL13/CXCR5/Tph] for the manufacture of a pharmaceutical composition for preventing or treating an immune-abnormal inflammatory disease, comprising a modulator of CXCL13, CXCR5, and/or peripheral helper T cells (Tph cells) [CXCL13/CXCR5/Tph].

### (Item D2)

The use according to the preceding item, wherein the modulator is selected from the group consisting of proteins, polypeptides, oligopeptides, peptides, polynucleotides, oligonucleotides, nucleotides, nucleic acids (including, for example, cDNA, DNA such as genomic DNA, and RNA such as mRNA), polysaccharides, oligosaccharides, lipids, organic small molecules (e.g., hormones, ligands, information transmitters, organic small molecules, molecules synthesized by combinatorial chemistry, small molecules that can be utilized as pharmaceuticals (e.g., small molecule ligands and the like), and the like), and complex molecules thereof.

### (Item D3)

The use according to any one of the preceding items, wherein the modulator is:
an anti-CXCL13 antibody, a nucleic acid medicine for CXCL13, or a CXCL13 inhibitor or peptide,
an anti-CXCR5 antibody, a nucleic acid medicine for CXCR5, or a CXCR5 inhibitor or peptide, or
an anti-Tph cell antibody, a nucleic acid medicine for Tph cell, or a Tph cell inhibitor or peptide.

### (Item D4)

The use according to any one of the preceding items, wherein the modulator is a modulator of CXCL13-CXCR5 interaction.

### (Item D5)

The use according to any one of the preceding items, wherein the modulator is a modulator of Tph cells.

### (Item D6)

The use according to any one of the preceding items, wherein the anti-CXCL13 antibody is a neutralizing antibody against CXCL13.

### (Item D7)

The use according to any one of the preceding items, wherein the anti-CXCL13 antibody is a chimeric antibody, humanized antibody, or human antibody against CXCL13.

### (Item D8)

The use according to any one of the preceding items, wherein the disease is a CXCL13-mediated disease.

### (Item D9)

The use according to any of the preceding items, wherein the disease comprises Castleman disease, rheumatoid arthritis, plasmacytosis, hyperimmunoglobulinemia, anemia, nephritis, cachexia, multiple myeloma, mesangial proliferative nephritis, systemic lupus erythematosus, systemic scleroderma, Crohn's disease, ulcerative colitis, pancreatitis, psoriasis, juvenile chronic arthritis, or systemic juvenile idiopathic arthritis, vasculitis, Kawasaki disease, TAFRO syndrome, and Poems syndrome.

### (Item D10)

The use according to any of the preceding items, wherein the disease comprises idiopathic multicentric Castleman disease.

### (Item E1)

A modulator of CXCL13, CXCR5, and/or peripheral helper T cells (Tph cells) [CXCL13/CXCR5/Tph] for use in prevention or treatment of an immune-abnormal inflammatory disease.

### (Item E2)

The modulator according to the preceding item, wherein the modulator is selected from the group consisting of proteins, polypeptides, oligopeptides, peptides, polynucleotides, oligonucleotides, nucleotides, nucleic acids (including, for example, cDNA, DNA such as genomic DNA, and RNA such as mRNA), polysaccharides, oligosaccharides, lipids, organic small molecules (e.g., hormones, ligands, information transmitters, organic small molecules, molecules synthesized by combinatorial chemistry, small molecules that can be utilized as pharmaceuticals (e.g., small molecule ligands and the like), and the like), and complex molecules thereof.

### (Item E3)

The modulator according to any one of the preceding items, wherein the modulator is:
an anti-CXCL13 antibody, a nucleic acid medicine for CXCL13, or a CXCL13 inhibitor or peptide,
an anti-CXCR5 antibody, a nucleic acid medicine for CXCR5, or a CXCR5 inhibitor or peptide, or
an anti-Tph cell antibody, a nucleic acid medicine for Tph cell, or a Tph cell inhibitor or peptide.

### (Item E4)

The modulator according to any one of the preceding items, wherein the modulator is a modulator of CXCL13-CXCR5 interaction.

### (Item E5)

The modulator according to any one of the preceding items, wherein the modulator is a modulator of Tph cells.

### (Item E6)

The modulator according to any one of the preceding items, wherein the anti-CXCL13 antibody is a neutralizing antibody against CXCL13.

### (Item E7)

The modulator according to any one of the preceding items, wherein the anti-CXCL13 antibody is a chimeric antibody, humanized antibody, or human antibody against CXCL13.

### (Item E8)

The modulator according to any one of the preceding items, wherein the disease is a CXCL13-mediated disease.

### (Item E9)

The modulator according to any of the preceding items, wherein the disease comprises Castleman disease, rheumatoid arthritis, plasmacytosis, hyperimmunoglobulinemia, anemia, nephritis, cachexia, multiple myeloma, mesangial proliferative nephritis, systemic lupus erythematosus, systemic scleroderma, Crohn's disease, ulcerative colitis, pancreatitis, psoriasis, juvenile chronic arthritis, or systemic juvenile idiopathic arthritis, vasculitis, Kawasaki disease, TAFRO syndrome, and Poems syndrome.

### (Item E10)

The modulator according to any of the preceding items, wherein the disease comprises idiopathic multicentric Castleman disease.

The compositions and methods of the present disclosure as described above can include any features described elsewhere herein.

In the present disclosure, it is intended that the one or more features may be provided in further combination in addition to the specified combination. Still further embodiments and advantages of the present disclosure will be appreciated by those skilled in the art upon reading and understanding the following detailed description if necessary.

### Advantages of the Invention

According to the present disclosure, a model animal such as a highly immunodeficient mouse (NOD-SCID/IL2Rnull) can be used to provide a Castleman disease patient tissue transplanted model animal. In addition, since it was shown that treatment using a modulator (e.g., inhibitor) of CXCL13, CXCR5, and/or peripheral helper T cells (Tph cells) [CXCL13/CXCR5/Tph] was successful in the model animal, it is possible to provide an effective therapeutic means for patients with Castleman disease and various diseases similar thereto.

### BRIEF DESCRIPTION OF THE DRAWINGS

FIG. 1 shows diagrams illustrating that xenotransplantation of LN cells of patients with iMCD-NOS resulted in fatal systemic inflammation in recipient mice.

FIG. 1A shows a scheme for xenotransplantation experiments.

FIG. 1B is survival analysis of recipient mice transplanted with LN cells of patients with iMCD-NOS (n=3) and controls (n=5). LN cells from identical patients were transplanted into at least 6 recipient mice. FIG. 1C shows representative photographs of iMCD-NOS NSG mice. Depilation of the whole body and auricular vasodilation were shown, respectively. FIG. 1D shows histological analysis results of spleens and livers of iMCD-NOS NSG (iMCD-NOS3).

FIG. 2 shows diagrams illustrating that plasmablasts capable of secreting human gamma-globulin proliferated when LN cells of patients with iMCD-NOS were xenotransplanted.

FIG. 2A shows representative FACS plots of cells in spleens of iMCD-NSG mice (iMCD-NOS2). FIG. 2B shows a graph showing significant body weight loss of iMCD-NSG mice after xenotransplantation of the LN cells of a patient with iMCD-NOS4. FIG. 2C shows graphs showing quantification of human IgG, IgA and IgE in sera of NSG mice transplanted with iMCD-NOS4 and control LN cells (Ctrl 1-3). *:p<0.05,**:p<0.01,***:p<0.001,****:p<0.0001

FIG. 3 shows experimental results showing that T-B interaction was required for generation of findings. FIG. 3A is a scheme for xenotransplantation experiments using unmanipulated LN cells and CD3⁺ T cell-depleted LN cells from two independent patients with iMCD NOS (iMCD-NOS2 and iMCD-NOS5). FIG. 3B shows graphs showing the percentage of human CD45⁺ hematopoietic cells in spleens of recipient mice transplanted with unmanipulated LN cells and CD3⁺ T cell-depleted LN cells at 8 weeks after xenotransplantation. FIG. 3C shows graphs showing quantification of human IgG, IgA, and IgE in sera of recipient mice transplanted with LN cells of the patient with iMCD-NOS2. FIG. 3D shows graphs quantifying human IgG, IgA, and IgE in sera of recipient mice transplanted with LN cells of the patient with iMCD-NOS5. FIG. 3E shows photographs confirming co-localization of hCD4⁺ T cells, hCD8⁺ T cells, and hCD20⁺ B cells in spleens and livers of iMCD-NOS NSG mice. *:p<0.05,**:p<0.01,***:p<0.001

FIG. 4 shows experimental results showing that proliferation of hCD4⁺hPD-1^{high}hCXCR5-hCCR2⁺ Tph cells was specifically observed in iMCD-NSG mice. FIG. 4A shows representative FACS plots of hCD45⁺ cells in spleens of iMCD-NSG mice (iMCD-NOS2). FIG. 4B is a graph showing the percentage of hPD-1^{high}hCXCR5-Tph cells within CD3⁺CD4⁺ human T cells in NSG mice and iMCD-NSG mice reconstituted with normal human hematopoiesis. FIG. 4C is a representative FACS plot of hCCR2 expression of hPD-1^{high}hCXCR5⁻ Tph cells in the spleens of iMCD-NSG mice (iMCD-NOS2). FIG. 4D shows a graph showing the percentage of PD-1^{high}CXCR5⁻ Tph cells in CD4⁺ T cells in reactive LN cells and iMCD-NOS LN cells (iMCD-NOS6) and a graph showing the percentage of PD-1^{high}CXCR5⁻ Tph cells in CD4⁺ T cells in PB from normal donors (n=3) and a patient with iMCD-NOS (iMCD-NOS6).

FIG. 4E shows results of heat map analysis of human (left) and mouse (right) cytokines and chemokines in sera of NSG mice in which human normal hematopoiesis was reconstituted by xenotransplantation of CB-derived CD34⁺HSPC (n=3) and iMCD-NSG5-derived LN cells (n=5). FIG. 4F shows results of comparing levels of human cytokines and chemokines between serum of original patient (iMCD-NOS5) and sera of iMCD-NSG mice transplanted with LN cells from identical patients. FIG. 4G is a representative intracellular FACS analysis result of CXCL13 expression in CD4+PD-1^{high}CXCR5⁻ Tph cells from the patient with iMCD-NOS (iMCD-NOS6). *:p<0.05,**:p<0.01,***:p<0.001,****:p<0.0001

FIG. 5 shows diagrams showing an improvement in survival of iMCD-NOS NSG mice when an anti-hCXCL13 antibody is administered. FIG. 5A is a scheme for in vivo therapeutic experiments by xenotransplantation of LN cells from two independent patients with iMCD-NOS (iMCD-NOS7 and iMCD-NOS8). FIG. 5B shows survival analysis results of iMCD-NSG mice treated with isotype IgG (n=10) and anti-hCXCL13 antibody (n=12). FIG. 5C shows survival analysis results of iMCD-NSG mice administered isotype IgG (n=12), rapamycin (n=12), and anti-hCXCL13 antibody (n=12). *:p<0.05,**:p<0.01,***:p<0.001

FIG. 6 shows survival analysis results showing that a lethal course of the recipient mice was improved by a CXCL13 neutralizing antibody. However, interruption of the CXCL13 neutralizing antibody exacerbates conditions, leading to cachexia and death.

### MODE FOR CARRYING OUT THE INVENTION

Hereinafter, the present disclosure will be described while showing the best mode. It should be understood that throughout the present specification, an expression in singular form also includes its concept in plural form, unless otherwise stated. Accordingly, it should be understood that a singular article (e.g., “a”, “an”, and “the” in English) also includes its concept in plural form, unless otherwise stated. In addition, it should also be understood that the terms used herein are used by the meanings which are commonly used in the art, unless otherwise stated. Accordingly, all technical terms and scientific terms used herein have the same meanings as those commonly understood by a person skilled in the art to which the present disclosure belongs, unless otherwise defined. In case of difference between the description of the present specification and the description of the art, the present specification (including definitions) is prioritized.

### Definitions

First, terms and general techniques used in the present disclosure will be described.

As used herein, “immune-abnormal inflammatory disease” refers to any disease that is caused by or considered to be caused by an immune abnormality and exhibits inflammatory conditions, and examples of the immune-abnormal inflammatory disease include a lymphoproliferative disease such as Castleman disease, an autoimmune disease, and an autoinflammatory disease.

Castleman disease (CD) begins in 1956 with Benjamin Castleman reporting a case of benign mediastinal lymph node enlargement characterized by lymphoid follicular hyperplasia and vascular hyperplasia. CD is histologically classified as hyaline-vascular type and plasma cell type. Castleman disease presents with systemic inflammation such as fever, anemia, elevation of acute phase proteins, and hypergammaglobulinemia, which is caused by IL-6 produced from enlarged lymph nodes (Yoshizaki K et al. Pathogenic significance of interleukin-6 (IL-6/BSF-2) in Castleman's disease. Blood. 1989 74(4): 1360-7). Hematologically, Castleman disease is classified as lymphoproliferative disease.

Castleman disease is divided into unicentric CD (UCD) and multicentric CD (MCD). UCD is a single regional lymph node lesion with Castleman-like pathology, often of the hyaline-vascular type, with mild inflammatory conditions relieved by surgical lymphadenectomy. MCD is often of the plasma cell type, with HHV8 (human herpes virus 8, also called KSHV: Kaposi's sarcoma herpesvirus) (HHV8-related MCD) and HHV8-negative idiopathic MCD (iMCD). Both are accompanied by multifocal lymph node enlargement and systemic inflammatory conditions having a characteristic pathology image, and are often considered as a differential disease of fever of unknown origin. HHV8-related MCD is observed with HIV infection and immunodeficiency, and virus IL-6 and human IL-6 encoded by HHV8 form pathology. Among multicentric Castleman disease, those of unknown cause in which human herpes virus type 8 infection is not observed are clearly distinguished and defined as idiopathic multicentric Castleman disease. Idiopathic multicentric Castleman disease exhibits clinical conditions such as fever, swollen lymph nodes, and anemia due to hyperinterleukin-6 syndrome, and often has a chronic course.

As used herein, CXCL13 and/or CXCR5 and/or peripheral helper T cells (Tph cells) are also referred to as CXCL13/CXCR5/Tph.

CXCL13 is a CXC chemokine also known as BLC (B lymphocyte chemoattractant) or BCA-1 (B cell-attracting chemokine 1), binds to receptor CXCR5, promotes migration of B lymphocytes, and has a function of inducing antibody production. This protein ligand can be observed in liver, lymph node, and spleen, and is expressed by neutrophils, macrophages, dendritic cells, and epithelial cells. Elevated levels of CXCL13 are expressed in tumors, particularly breast cancer patients with metastatic disease. Human mRNA can be identified by IDs: NM 006419 and NM 001371558, and NM 018866 is known in mice. As amino acid sequences of proteins, NP_006410 and NP_001358487 are known in humans, and NP 061354 is known in mice.

CXCR5 is an abbreviation for C—X—C chemokine receptor type 5 (CXC-R5), and is also referred to as CD185 or Burkitt lymphoma receptor 1 (BLR1). CXCR5 is a G-protein seven-transmembrane receptor, which is the only receptor for CXCL13. CXCR5 is also a member of the CXC chemokine receptor and migrates T cells to lymph nodes and B-cell zone. As sequences of mRNA, NM_032966 and NM_001716 are known for humans, and NM 007551 is known in mice. As amino acid sequences of proteins, NP_001707 and NP_116743 are known in humans, and NP 031577 is known in mice.

The peripheral helper T cell (Tph cell) refers to a helper T cell having a characteristic that expression of a molecule on the surface of the cell is PD-1^{high} CXCR5 (+) ICOS (+).

As used herein, a “modulator” refers to any agent capable of modulating the activity or the like of a substance such as a certain target molecule or a cell or the like with respect to a subject such as the molecule or the cell or the like. A modulator can usually exert its function by interacting with a target.

Modulators generally refer to any inhibitory, suppression, or activating compound identified by in silico, in vitro and/or in vivo assays for a substance or entity comprising the same, including, for example, agonists, antagonists, and fragments, variants, and mimetics thereof. Here, modulation means increase or decrease, enhancement or suppression (or elimination), or the like.

As used herein, “decrease” or “suppression” of activity or expression product (e.g., protein, transcript (RNA or the like)) or synonyms thereof refers to a decrease in the quantity, quality, or effect of a specific activity, transcript, or protein, or activity that decreases the same. Among decrease, “elimination” refers to activity, expression product, or the like being less than the detection limit and is especially referred to as “elimination”. As used herein, “elimination” is encompassed by “decrease” or “suppression”.

As used herein, “increase” or “activation” of activity or expression product (e.g., protein, transcript (RNA or the like)) or synonyms thereof refers to an increase in the quantity, quality, or effect of a specific activity, transcript, or protein, or activity that increases the same.

As used herein, an “agent” may be any substance or another element (e.g., energy such as light, radioactivity, heat, or electricity) as long as the intended purpose can be achieved. Examples of such a substance include, but are not limited to, proteins, polypeptides, oligopeptides, peptides, polynucleotides, oligonucleotides, nucleotides, nucleic acids (including, for example, cDNA, DNA such as genomic DNA, and RNA such as mRNA), polysaccharides, oligosaccharides, lipids, organic small molecules (e.g., hormones, ligands, information transmitters, organic small molecules, molecules synthesized by combinatorial chemistry, small molecules that can be utilized as pharmaceuticals (e.g., small molecule ligands and the like), and the like), and complex molecules thereof. In the present disclosure, a substance that binds to CXCL13, CXCR5, and/or peripheral helper T cells (Tph cells) [as used herein, it is also referred to as CXCL13/CXCR5/Tph] may also be such an agent. Typical examples of an agent specific to a polynucleotide include, but are not limited to, a complementary polynucleotide having a certain sequence homology (e.g., 70% or more sequence identity) to the sequence of the polynucleotide, a polypeptide such as a transcription factor that binds to a promoter region, and the like. Typical examples of an agent specific to a polypeptide include, but are not limited to, an antibody directed specifically to the polypeptide, or a derivative thereof or an analogue thereof (e.g., single-chain antibody), a specific ligand or receptor in the case where the polypeptide is a receptor or ligand, and a substrate and the like in the case where the polypeptide is an enzyme.

As used herein, “interaction” means that each of two substances exert a force (e.g., intermolecular force (van der Waals force), hydrogen bond, or hydrophobic interaction) between one substance and other substance. In general, two interacting substances are associated with or linked to each other. The modulator of the present disclosure can be realized by utilizing such interaction. As used herein, an “agent” (or detection agent or the like) that “specifically” interacts with (or binds to) a subject encompasses agents with affinity to the subject that is typically similar or higher, or preferably significantly (e.g., statistically significantly) higher than affinity to other unrelated entities (e.g., when the subject is a polynucleotide or polypeptide, a polynucleotide or polypeptide with less than 30% identity). Such affinity can be measured, for example, by hybridization assay, binding assay, or the like.

As used herein, a first substance or agent “specifically” interacting with (or binding to) a second substance or agent refers to the first substance or agent interacting with (or binding to) the second substance or agent at a higher affinity than with substances or agents other than the second substance or agent (especially other substances or agents in a sample containing the second substance or agent). Examples of an interaction (or bond) specific to a substance or agent include, but are not limited to, hybridization in a nucleic acid, antigen-antibody reaction in a protein, enzyme-substrate reaction, nucleic acid-protein reaction, protein-lipid interaction, nucleic acid-lipid interaction, and the like. Thus, when substances or agents are both nucleic acids, a first substance or agent “specifically interacting” with a second substance or agent encompasses the first substance or agent having at least partial complementarity to the second substance or agent. Further, for example, when substances or agents are both proteins, examples of a first substance or agent “specifically” interacting with (or binding to) a second substance or agent include, but are not limited to, interaction by an antigen-antibody reaction, interaction by a receptor-ligand reaction, enzyme-substrate interaction, and the like. When two types of substances or agents include a protein and a nucleic acid, a first substance or agent “specifically” interacting with (or binding to) a second substance or agent encompasses an interaction (or a bond) between an antibody and an antigen thereof. Such a specific interaction or binding reaction can be utilized to detect or quantify a target in a sample.

The modulator of the present disclosure is capable of modulating expression (in the case of a polynucleotide, a polypeptide or the like) in a subject. As used herein, detection or quantification of polynucleotide or polypeptide expression can be achieved using a suitable method, including, for example, measurement of mRNAs and an immunological measurement method, including a bond to or interaction with a detection agent, inspection agent or diagnostic agent (including application as a companion reagent). Examples of a molecular biological measurement method include Northern blot, dot blot, PCR, and the like. Examples of the immunological measurement method include ELISA using a microtiter plate, RIA, fluorescent antibody method, luminescence immunoassay (LIA), immunoprecipitation (IP), single radial immunodiffusion (SRID), turbidimetric immunoassay (TIA), Western blot, immunohistochemical staining, and the like. Further, examples of a quantification method include ELISA, RIA, and the like. Quantification may also be performed by a gene analysis method using an array (e.g., DNA array, protein array). DNA arrays are outlined extensively in (Ed. by Shujunsha, Saibo Kogaku Bessatsu “DNA Maikuroarei to Saishin PCR ho” [Cellular engineering, Extra issue, “DNA Microarrays and Latest PCR Methods” ]). Protein arrays are described in detail in Nat Genet. 2002 December; 32 Suppl: 526-532. Examples of the method for analyzing gene expression include, but are not limited to, RT-PCR, RACE, SSCP, immunoprecipitation, two-hybrid system, in vitro translation, and the like, in addition to the methods described above. Such additional analysis methods are described in, for example, Genomu Kaiseki Jikkenho Nakamura Yusuke Labo Manyuaru [Genome analysis experimental method Yusuke Nakamura Lab Manual], Ed. by Yusuke Nakamura, Yodosha (2002) and the like, which is incorporated herein by reference in its entirety.

As used herein, “expression” of a gene, a polynucleotide, a polypeptide, or the like refers to the gene or the like being subjected to a certain action in vivo to be converted into another form. Preferably, expression refers to a gene, a polynucleotide, or the like being transcribed and translated into a form of a polypeptide. However, transcription to produce mRNA is also one embodiment of expression. Thus, “expression product” as used herein encompasses such a polypeptide or protein, or mRNA. As used herein, a “gene” refers to an agent that defines a genetic trait, and a “gene” may refer to a “polynucleotide”, an “oligonucleotide”, and a “nucleic acid”.

As used herein, “expression level” refers to the amount of polypeptide, mRNA, or the like that is expressed in a cell, tissue, or the like of interest. When the modulator of the present disclosure is, for example, an antibody, such expression level is assessed by any suitable method including an immunological measurement method such as ELISA, RIA, fluorescent antibody method, Western blot, or immunohistochemical staining by using the antibody. Examples of the expression level include the expression level of the polypeptide of the present disclosure at a protein level, or the expression level of the polypeptide used in the present disclosure at an mRNA level assessed by any suitable method including a molecular biological measurement method such as Northern blot, dot blot, or PCR. “Change in expression level” refers to an increase or decrease in the expression level of the polypeptide used in the present disclosure at a protein level or mRNA level assessed by any suitable method including the immunological measurement method or the molecular biological measurement method. By measuring the expression level of a certain marker, a variety of detection or diagnosis based on the marker can be performed.

As used herein, an “antibody” includes a molecule capable of specifically binding to a specific epitope on an antigen or a population thereof. An antibody may also be a polyclonal antibody or a monoclonal antibody. An antibody can have various forms such as one or more forms selected from the group consisting of full length antibodies (antibodies with a Fab region and an Fc region), Fv antibodies, Fab antibodies, F(ab′)2 antibodies, Fab′ antibodies, diabodies, single-stranded (single chain) antibodies (e.g., scFv), sc(Fv)2 (single chain (Fv)2), scFv-Fc, dsFv, multispecific antibodies (e.g., oligospecific antibodies and bispecific antibodies), diabodies, peptides or polypeptides with antigen binding properties, chimeric antibodies (e.g., mouse-human chimeric antibodies, chicken-human chimeric antibodies, and the like), mouse antibodies, chicken antibodies, humanized antibodies, human antibodies, and equivalents thereof. An antibody also encompasses a modified antibody or an unmodified antibody. The modified antibody may be formed by an antibody binding to various molecules such as polyethylene glycol. The modified antibody can be obtained by applying chemical modification to an antibody using a known method. Such an antibody can also be covalently bound, or fused by recombination, to an enzyme such as alkaline phosphatase, horseradish peroxidase, or a galactosidase. The anti-CXCL13, CXCR5, and/or peripheral helper T cell (Tph cell) antibody used in the present disclosure may bind to a substance such as a CXCL13 protein, a CXCR5 protein, and/or a protein contained in the peripheral helper T cells (Tph cells), and the source, type, shape, and the like thereof are not limited. Specifically, a known antibody such as an antibody from a non-human animal (e.g., a mouse antibody, a rat antibody, and a camel antibody), a human antibody, a chimeric antibody, or a humanized antibody can be used. In the present disclosure, a monoclonal or polyclonal antibody can be used, but a monoclonal antibody is preferable. It is preferable that an antibody binds specifically to CXCL13, CXCR5, and/or peripheral helper T cells (Tph cells). An antibody also encompasses a modified antibody or an unmodified antibody. The modified antibody may be formed by an antibody binding to various molecules such as polyethylene glycol. The modified antibody can be obtained by applying chemical modification to an antibody using a known method. An antibody may preferably be a “neutralizing antibody”. A “neutralizing antibody” refers to an antibody in which, after the antibody binds to a subject, at least one of activities such as biological activity, preferably biological activity, of the subject is inhibited or eliminated.

As used herein, the term “activity” refers to a function of a molecule in the broadest sense. Activity generally encompasses, but is not intended to be limited to, biological function, biochemical function, physical function, and chemical function of a molecule. Examples of activity include enzymatic activity, ability to interact with another molecule, ability to activate, promote, stabilize, inhibit, suppress, or destabilize a function of another molecule, stability, and ability to localize at a specific position in a cell. Where applicable, the term also relates to a function of a protein complex in the broadest sense.

As used herein, “biological function” or “biological activity”, with regard to a gene or a nucleic acid molecule or polypeptide related thereto, refers to a specific function or activity that the gene, nucleic acid molecule or polypeptide can have in vivo, and examples thereof include, but are not limited to, production of a specific antibody, enzymatic activity, impartation of resistance, and the like. In the present disclosure, examples thereof include, but are not limited to, functions involved in CXCL13, CXCR5, and/or peripheral helper T cells (Tph cells) (e.g., functions involved in Tph cells including floating and activation of B cells, formation of lymph follicles ectopically with inflammation, contribution to generation of tertiary lymphoid structures (TLSs) in inflamed tissues, and association with infectious diseases, autoimmune diseases, and malignant tumors, and the like, and functions involved in CXCL13 including stimulation of CXCR5 of GPCR as a ligand, and contribution to migration of cells via inflow of intracellular Ca²⁺ and ERK/MAPK signals, involvement in proliferation and survival of breast cancer via activation of ERK signals and cyclin D1, reduction of ERK activity in breast cancer by treatment with CXCL13 antibody in vivo, and association with pathogenesis of many solid tumors such as non-small cell lung cancer, pancreatic cancer, liver cancer, and gastric cancer, and the like), and the like. As used herein, biological function can be exerted by “biological activity”. The “biological activity” as used herein refers activity that a certain agent (e.g., polynucleotide, protein, or the like) can have in vivo, including activity exerting a variety of functions (e.g., transcription promoting activity) such as the activity of activating or deactivating a molecule from interaction with a certain molecule. When two agents interact, the biological activity thereof can be the bond between the two molecules and the biological change resulting therefrom, and for example, it is considered that the two molecules are bound when precipitation of one of the molecules with an antibody results in co-precipitation of the other molecule. Thus, one method of determination includes observing such co-precipitation. For example, when an agent is an enzyme, the biological activity thereof encompasses the enzymatic activity thereof. In another example, when an agent is a ligand, the biological activity thereof encompasses binding of the ligand to a corresponding receptor. Such biological activity can be measured by a technology well known in the art. Thus, “activity” refers to various measurable indicators that indicate or reveal the bond (either directly or indirectly) or affect a response (i.e., having a measurable effect in response to some exposure or stimulation). Examples thereof include affinity of a compound that directly binds to the polypeptide or polynucleotide of the present invention, the amount of proteins upstream or downstream after some stimulation or event, and a scale of another similar function.

The modulator and the like of the present disclosure can be isolated or purified and provided. As used herein, a “purified” substance or biological agent (e.g., nucleic acid, protein, or the like) refers to a substance or biological agent having at least a part of an agent that naturally accompanies the substance or biological agent removed. Thus, the purity of a substance or biological agent in a purified substance or biological agent is generally higher (i.e., concentrated) than the purity in the normal state of the substance or biological agent. The term “purified” as used herein refers to the presence of preferably at least 75% by weight, more preferably at least 85% by weight, still more preferably at least 95% by weight, and most preferably at least 98% by weight of a biological agent of the same type. The substance or biological agent used in the present invention is preferably a “purified” substance or biological agent. An “isolated” substance or biological agent (e.g., nucleic acid, protein, or the like) as used herein refers to a substance or biological agent having an agent that naturally accompanies the substance or biological agent substantially removed. Since the term “isolated” as used herein varies depending on the objective, the term does not necessarily have to be expressed in purity. However, when necessary, the term refers to the presence of preferably at least 75% by weight, more preferably at least 85% by weight, still more preferably at least 95% by weight, and most preferably at least 98% by weight of a substance or biological agent of the same type. The substance used in the present invention is preferably an “isolated” substance or biological agent.

As used herein, “functional equivalent” refers to any entity having the same target function but a different structure relative to the original target entity. Thus, it is understood that a functional equivalent of “CXCL13, CXCR5, and/or peripheral helper T cells (Tph cells)” or an antibody thereof encompasses mutants or variants (e.g., amino acid sequence variants or the like) of CXCL13, CXCR5, and/or peripheral helper T cells (Tph cells) or antibody thereof that are not CXCL13, CXCR5, and/or peripheral helper T cells (Tph cells) or antibody thereof itself, which have the biological action of CXCL13, CXCR5, and/or peripheral helper T cells (Tph cells) or antibody thereof or can change, upon action, into CXCL13, CXCR5, and/or peripheral helper T cells (Tph cells) or the antibody thereof itself, or a mutant or variant of CXCL13, CXCR5, and/or peripheral helper T cells (Tph cells) or the antibody thereof (e.g., including nucleic acids encoding CXCL13, CXCR5, and/or peripheral helper T cells (Tph cells) or an antibody thereof itself and mutants or variants of CXCL13, CXCR5, and/or peripheral helper T cells (Tph cells) or antibody thereof, and vectors, cells, and the like comprising such a nucleic acid). In the present disclosure, it is understood, even without specifically mentioning, that a functional equivalent of CXCL13, CXCR5, and/or functional equivalents of peripheral helper T cells (Tph cells) or an antibody thereof can be used in the same manner as CXCL13, CXCR5, and/or functional equivalents of peripheral helper T cells (Tph cells) or antibody thereof. A functional equivalent can be found by searching a database or the like. As used herein, “search” refers to utilizing a certain nucleic acid base sequence electronically, biologically, or by another method to find another nucleic acid base sequence having a specific function and/or property. Examples of electronic search include, but are not limited to, BLAST (Altschul et al., J. Mol. Biol. 215: 403-410 (1990)), FASTA (Pearson & Lipman, Proc. Natl. Acad. Sci., USA 85: 2444-2448 (1988)), Smith and Waterman method (Smith and Waterman, J. Mol. Biol. 147: 195-197 (1981)), Needleman and Wunsch method (Needleman and Wunsch, J. Mol. Biol. 48: 443-453 (1970)), and the like. Examples of biological search include, but are not limited to, stringent hybridization, a macroarray with a genomic DNA applied to a nylon membrane or the like or a microarray with a genomic DNA applied to a glass plate (microarray assay), PCR, in situ hybridization, and the like. As used herein, a gene used in the present disclosure is intended to include corresponding genes identified by such electronic search or biological search.

The modulator of the present disclosure can be a substance that binds to CXCL13, CXCR5, and/or peripheral helper T cells (Tph cells), a CXCL13, CXCR5, and/or peripheral helper T cell (Tph cell) binder, or a CXCL13, CXCR5, and/or peripheral helper T cell (Tph cell) interaction molecule, which is a molecule or substance that binds to CXCL13, CXCR5, and/or peripheral helper T cells (Tph cells) at least temporarily. It is preferably and advantageously capable of displaying (e.g., labeled or in a labelable state) that the molecule or substance is bound for detection purposes, and is advantageously further bound to a therapeutic agent for therapeutic purposes. Examples of a substance that binds to CXCL13, CXCR5, and/or peripheral helper T cells (Tph cells) include antibodies, binding peptides, peptidomimetics, and the like. Preferably, a substance that binds to CXCL13, CXCR5, and/or peripheral helper T cells (Tph cells) has intracellular invasion (internalization) activity. As used herein, a “binding protein” or “binding peptide” with respect to CXCL13, CXCR5, and/or peripheral helper T cells (Tph cells) refers to any protein or peptide that binds to CXCL13, CXCR5, and/or peripheral helper T cells (Tph cells) including, but not limited to, antibodies directed to CXCL13, CXCR5, and/or peripheral helper T cells (Tph cells) (e.g., polyclonal antibody or monoclonal antibody), antibody fragments, and functional equivalents.

The medicament of the present disclosure may be provided as a kit. As used herein, a “kit” refers to a unit generally providing portions to be provided (e.g., inspection drug, diagnostic drug, therapeutic drug, antibody, label, manual, and the like) in two or more separate sections. This form of a kit is preferred when a composition that should not be provided in a mixed state and is preferably mixed immediately before use for safety reasons or the like is intended to be provided. Such a kit advantageously comprises an instruction or manual describing how the provided portions (e.g., inspection drug, diagnostic drug, or therapeutic drug are used or how a reagent should be handled. When the kit is used herein as a reagent kit, the kit generally comprises an instruction describing how to use an inspection drug, diagnostic drug, therapeutic drug, antibody, and the like.

As used herein, “therapy” refers to prevention of exacerbation, preferably maintaining of the current state, more preferably alleviation, and still more preferably elimination of a disease or disorder (e.g., immune-abnormal inflammatory disease) when such a condition has developed, including being capable of exerting a condition-improving effect or a prophylactic effect of a disease of a patient or one or more conditions accompanying the disease. Preliminary diagnosis with suitable therapy may be referred to as “companion therapy” and a diagnostic drug therefor may be referred to as “companion diagnostic agent”.

As used herein, “prevention” refers to preventing a disease or disorder (e.g., immune-abnormal inflammatory disease) from being in such a condition prior to being in such a condition. For example, it is possible to use the agent of the present disclosure to perform diagnosis, and optionally use the agent of the present disclosure to prevent or take measures to prevent immune-abnormal inflammatory disease or the like.

The present disclosure may provide a therapeutic drug of immune-abnormal inflammatory disease and the like. As used herein, a “therapeutic drug (agent)” broadly refers to all agents capable of treating a condition of interest (e.g., disease such as immune-abnormal inflammatory disease or the like). In one embodiment of the present disclosure, the “therapeutic drug” may be a pharmaceutical composition comprising an active ingredient and one or more pharmacologically acceptable carriers. The pharmaceutical composition can be manufactured, for example, by mixing an active ingredient and the carriers by any method known in the technical field of pharmaceuticals. Further, usage mode of a therapeutic drug is not limited, as long as it is used for therapy. A therapeutic drug may be the active ingredient alone or a mixture of an active ingredient and any ingredient. Furthermore, the shape of the carriers is not particularly limited, and may be, for example, a solid or liquid (e.g., buffer solution). It should be noted that a therapeutic drug of immune-abnormal inflammatory disease may broadly include a drug (prophylactic drug) for preventing immune-abnormal inflammatory disease.

The present disclosure may provide a prophylactic drug of immune-abnormal inflammatory disease and the like. As used herein, a “prophylactic drug (agent)” broadly refers to all agents capable of preventing a condition of interest (e.g., disease such immune-abnormal inflammatory disease or the like).

The medicament of the present disclosure may be provided with instructions. As used herein, “instruction” is a document explaining the method of use of the present disclosure for a physician or other users, and may be referred to as a package insert, a label, or the like. The instruction has a description of the detection method of the present disclosure, method of use of a diagnostic agent, or instruction to administer a medicament or the like. Further, an instruction may have a description instructing oral or intravenous administration (e.g., by injection or the like) as a site of administration. The instruction is prepared in accordance with a format specified by the regulatory agency of the country in which the present disclosure is practiced (e.g., the Ministry of Health, Labor and Welfare in Japan, Food and Drug Administration (FDA) in the U.S., or the like), with an explicit description showing approval by the regulatory agency. The instruction is a so-called package insert and is typically provided in, but not limited to, paper media, and can also be provided in a form such as an electronic medium (e.g., a web site provided on the Internet or an email).

As used herein, a “model animal” refers to an experimental animal that simulates or develops a disease in a certain animal (particularly, human), and examples thereof include animals that develop various diseases such as immune-abnormal inflammatory disease (e.g., Castleman disease). In the present disclosure, an animal as a model of an immune-abnormal inflammatory disease can be produced by transplanting lymph node cells other than the immunodeficient animal in an immunodeficient animal (e.g., immunodeficient mouse) as a model animal. Therefore, in the present disclosure, such an animal can be mentioned as an example of the model animal. Examples of the model animal include any animal, and mammals and animals similar to humans are preferable. For example, rodents, primates, and the like are generally used, but the model animal is not limited thereto, and specific examples thereof include, but not limited to, mice, rats, rhesus monkeys, and the like.

As used herein, an “immunodeficient” animal refers to an animal that lacks at least a part of (or lacks all of) immune functions that are usually possessed (e.g., B cell function, T cell function, NK activity, complement activity, macrophage function, lymph node, Peyer's patch, and the like), and examples thereof include T cell function-deficient animals (e.g., Balb/cAJcL nu/nu mouse), B cell/T cell function-deficient animals (e.g., C.B-17 lcr-scid/scidJcl mouse, etc.), B cell/T cell function deficiency, NK activity deficiency, complement activity deficiency, macrophage function-deficient animals, lymph node/Peyer's patch-deficient animals, and the like (e.g., CLEA Japan, and the like, https://www.clea-japan.com/products/animal/immunodeficiency).

As used herein, a “lymph node cell” refers to a cell present in a lymph node. The lymph node includes lymphatic sinuses composed of reticular tissues and lymph nodules where lymphocytes (cells that produce immune antibodies) gather. The lymph node has a cortex and a medulla, and further the cortex has a follicle composed of B cells in the center and a paracortex composed mainly of T cells in the periphery. In the present disclosure, examples of the lymph node cell include lymph node cells of a patient with Castleman disease, for example, a patient with non-specific multicentric Castleman disease.

### Preferred Embodiments

Hereinafter, preferred embodiments of the present disclosure will be described. Embodiments described below are provided to facilitate the understanding of the present disclosure, and it is understood that the scope of the present disclosure should not be limited to the following descriptions. Thus, it is apparent that those skilled in the art can make appropriate modifications within the scope of the present disclosure by referring to the descriptions herein. It is also understood that the following embodiments of the present disclosure can be used alone or in combination.

### (Pharmaceutical Composition)

The present disclosure provides compositions for preventing or treating an immune-abnormal inflammatory disease, comprising a modulator of CXCL13, CXCR5, and/or peripheral helper T cells (Tph cells) [CXCL13/CXCR5/Tph]. In one embodiment, the immune-abnormal inflammatory disease can include a lymphoproliferative disease (e.g., Castleman disease).

Castleman disease (CD) is a lymphoproliferative disease characterized by swollen lymph nodes, hypergammaglobulinemia, hepatosplenomegaly, and systemic inflammation. CD is classified into at least two distinct diseases: unicentric CD (UCD) and multicentric CD (MCD).

These two types of CD have different clinical characteristics and prognoses, and MCD is known to have a worse prognosis than UCD. Furthermore, MCD is classified into idiopathic MCD (iMCD), human herpes virus 8 (HHV-8)-related MCD (HHV8-MCD), and polyneuropathy, organomegaly, endocrinopathy, monoclonal plasma cell disorders, skin changes (POEMS)-related MCD (POEMS-MCD).

In addition, iMCD is classified into two distinct subsets: a subtype of TAFRO (thrombocytopenia, anasarca, fever, reticulin fibrosis or renal dysfunction, organomegaly) iMCD (iMCD-TAFRO) and a subtype of non-specific iMCD (iMCD-NOS).

The present disclosers have demonstrated that IL-6 is an important disease driver for CD. Therefore, for the treatment of iMCD, an anti-IL-6 therapy such as siltuximab and tocilizumab or an anti-IL-6 receptor therapy is a standard therapy. However, it is known that about ½ of iMCD patients do not respond to IL-6 inhibition therapy, and cases resistant to IL-6 inhibition are suspected of involvement of alternative cytokines or chemokines.

In iMCD-TAFRO, it is known that an mTOR signal is enhanced in lymph node tissue of a CD patient with IL-6 inhibitor resistance. Transcription studies have also revealed that type 1 IFN signal enhances JAK-dependent mTOR activation, thereby contributing to the pathogenesis of iMCD-TAFRO. On the other hand, data on the pathogenesis of iMCD-NOS is limited, due to a low incidence of iMCD-NOS and the absence of an appropriate animal experimental model of iMCD. In one embodiment of the present disclosure, use of a patient-derived xenograft (PDX) model allows evaluating the efficacy of a new therapeutic strategy against Castleman disease.

In the present disclosure, as described in Examples below, it was found that when human LN cells purified from a patient with iMCD-NOS were xenotransplanted, a disease state similar to iMCD such as hypergammaglobulinemia or hypercytokinemia was reproduced, and fatal inflammation occurred in vivo. Furthermore, since removal of human CD3⁺ T cells from the graft did not completely develop iMCD-like inflammation, it was found that interaction between human T cells and B cells plays an essential role in the development of iMCD-like systemic inflammation in vivo. Interestingly, there was a significant increase in CXCL13 that was elevated upon “flare” of iMCD when compared to patient serum data. Furthermore, blocking CXCL13 using a neutralizing antibody significantly ameliorated lethal inflammation in mice transplanted with iMCD-NOS LN cells.

From the above, in one aspect of the present disclosure, a PDX model of iMCD is provided, and CXCL13 produced by Tph cells plays a critical role in the development of iMCD-like disease states, and therefore a therapy targeting CXCL13 in the treatment of iMCD-NOS and a composition containing a modulator of CXCL13 are provided.

In one embodiment of the present disclosure, the immune-abnormal inflammatory disease includes Castleman disease, rheumatoid arthritis, plasmacytosis, hyperimmunoglobulinemia, anemia, nephritis, cachexia, multiple myeloma, mesangial proliferative nephritis, systemic lupus erythematosus, systemic scleroderma, Crohn's disease, ulcerative colitis, pancreatitis, psoriasis, juvenile chronic arthritis, or systemic juvenile idiopathic arthritis, vasculitis, Kawasaki disease, TAFRO syndrome, and Poems syndrome. In one embodiment, the immune-abnormal inflammatory disease can include idiopathic multicentric Castleman disease. In one embodiment, the immune-abnormal inflammatory disease may be a CXCL13-mediated disease.

In one embodiment of the present disclosure, the composition of the present disclosure is also effective against cancer expressing CXCR5.

In one embodiment of the present disclosure, the modulator of CXCL13, CXCR5, and/or peripheral helper T cells (Tph cells) [CXCL13/CXCR5/Tph] can be selected from the group consisting of proteins, polypeptides, oligopeptides, peptides, polynucleotides, oligonucleotides, nucleotides, nucleic acids (including, for example, cDNA, DNA such as genomic DNA, and RNA such as mRNA), polysaccharides, oligosaccharides, lipids, organic small molecules (e.g., hormones, ligands, information transmitters, organic small molecules, molecules synthesized by combinatorial chemistry, small molecules that can be utilized as pharmaceuticals (e.g., small molecule ligands and the like), and the like), and complex molecules thereof. Such a modulator may be known modulator, or can be obtained by a screening method as described later.

In one embodiment of the present disclosure, the modulator of CXCL13, CXCR5, and/or peripheral helper T cells (Tph cells) [CXCL13/CXCR5/Tph] can be (1) an anti-CXCL13 antibody, a nucleic acid medicine for CXCL13, or a CXCL13 inhibitor or peptide, (2) an anti-CXCR5 antibody, a nucleic acid medicine for CXCR5, or a CXCR5 inhibitor, or (3) an anti-Tph cell antibody, a nucleic acid medicine for Tph cells, or a Tph cell inhibitor. Examples of a transcription factor that is a positive regulator of CXCL13 include Sox4 (Yoshitomi H. et al. Nat Commun. 2018 Sep. 19; 9(1): 3762., Kobayashi S. et al. Eur J Immunol. 2016 February; 46(2): 360-71). In addition, examples of a transcription factor of a negative regulator of CXCL13 include Blimp1. In another embodiment, IL-21, IL-23, IL-6, or type 1 IFN can also be used as a control factor of CXCL13.

In one embodiment of the present disclosure, an anti-CXCL13 antibody can be used as a modulator of CXCL13, and the anti-CXCL13 antibody can be a neutralizing antibody against CXCL13. Examples of the neutralizing antibody against CXCL13 include Biolegend Ultra-LEAF (trademark) Purified anti-human CXCL13 (BLC) antibody (Catalog no. 934503), Ultra-LEAF: Purifide anti-human CXCL13 antibody (Biolegend, cat #934506, mouse IgG1, k), PetroTeck (cat #300-47), BMC anti-CXCL13ab, and the like. In another embodiment, the anti-CXCL13 antibody can also be a chimeric antibody, humanized antibody, or human antibody against CXCL13. The chimeric antibody, humanized antibody, or human antibody can be produced from known anti-CXCL13 antibodies, including the above antibodies, using various methods well known in the art.

In one embodiment of the present disclosure, the modulator of CXCL13, CXCR5, and/or peripheral helper T cells (Tph cells) [CXCL13/CXCR5/Tph] may be a modulator of CXCL13-CXCR5 interaction. For example, among the transcription factors and control factors mentioned above, those involved in the CXCL13-CXCR5 interaction can also be used.

Also, in one embodiment of the present disclosure, the modulator of CXCL13, CXCR5, and/or peripheral helper T cells (Tph cells) [CXCL13/CXCR5/Tph] may be a modulator of Tph cells.

Peripheral helper T cells (Tph cells) and follicular helper T cells (Tfh cells) have the following common points and differences in diseases.

| | Differentiation | Differentiation | Differentation | CXCR5 | Mature Th | Production | Target B | |
|---|---|---|---|---|---|---|---|---|
| | origin | induction | [symbol] | expression | surface | factor | cell | Location |
| Tfh | CD4 [symbol] T | TGF-β | IL-12/23 | + | PD-1⁺ | IL-21 | NaiveB | Lymph node |
| (Lymph node} | | ActivinA | | (CXCR5⁻) | CXCR5⁻ | CXCL-13 | (CXCR5⁺) | Embryo |
| | | | | | ICOS⁺ | | MemoryB | (Lymph node) |
| | | | | | BCL-6 | | (CXCR5⁺) | |
| Tph | CD4 [symbol] T | TGF-β | Cytokines(?) | − | PD-1⁺ | IL-21 | MemoryB | Ectopic lymphoid |
| (Site of | | ActivinA | IL-6 | (CXCR5⁻) | CXCR5⁻ | CXCL-13 | (CXCR5⁻) | follicle |
| inflammation) | | | TNF-α | | CCR2⁺ | | Atypical | (Site of |
| | | | IL-12/23 | | ICOS⁺ | | MemoryB | inflammation) |
| | | | Type I IFN | | CCR5⁺ | | (CXCR5⁻) | (Autoantibody) |
| | | | | | SOX4 | | | |
| | | | | | CXCR1 | | | |

[symbol]

indicates data missing or illegible when filed

Both Tfh and Tph act on CXCR5⁺ B cells via CXCL13. Tph forms ectopic lymphoid follicles as B cell inducers and acts on CD11c⁺CD21⁻CXCR5⁻ B cells to increase production of autoantibodies (e.g. in the case of SLE). On the other hand, Tfh acts on change from IgM to IgA to IgG in isotype shifting. Therefore, without being bound by theory, it is considered that the inhibition of CXCL13 suppresses the activity of B cells, and in particular, Tph-B signal inhibition makes it possible to suppress the production of autoantibodies, thereby making it possible to treat SLE and RA.

In the case of peripheral Tph cell-increasing diseases such as Sjögren's syndrome, IgG4-RA, scleroderma, IgA nephropathy, type I diabetes, RA, and SLE, Tph cells and CXCL13 can be biomarkers for diagnosis and exacerbation of the diseases. Tph cells activate CXCR5⁺ Tfh cells, increase inflammation by inducing production of CXCL13, and activate B cells. There is also a possibility that Tph-Tfh is mutually differentiated (Cell Mol Immunol. 2021 March; 18 (3): 523-527.).

In addition, since it has attracted attention that Tph cells are involved in the pathogenesis of autoimmune diseases such as RA and SLE (Front Immunol. 2018; 9: 1924., Rheumatology 2019 Sep. 1; 58(9): 1662-1673., Ann Rheum Dis. 2019 Oct; 78(10): 1346-1356., Semin Arthritis Rheum. 2020 Oct; 50(5): 867-872., JCI Insight. 2019 Oct. 17; 4(20): e130062., Curr Opin Rheumatol. 2019 January; 31(1): 9-15. etc.), it is considered that in one embodiment of the present disclosure, by using a CXCL13 inhibitor or a CXCR5 inhibitor, it is possible to treat autoimmune diseases such as RA and SLE via Tph-B signal inhibition and Tph-Tfh inhibition.

### (Model Animal)

In one aspect of the present disclosure, an immune-abnormal inflammatory disease model animal is provided. In one embodiment, the model animal of the present disclosure can be a model animal of Castleman disease, preferably idiopathic multicentric Castleman disease. In one embodiment, the animal is an immunodeficient animal and contains lymph node cells derived from other than the immunodeficient animal. The lymph node cells used are not particularly limited as long as they are cells of a patient with an immune-abnormal disease, but in one embodiment, the lymph node cells may be lymph node cells of a patient with iMCD-NOS.

The immune-abnormal inflammatory disease model animal of the present disclosure can be produced by xenotransplantation into a non-human animal a graft derived from a patient suffering from an immune-abnormal inflammatory disease. Therefore, in one aspect of the present disclosure, there is provided a method for producing an immune-abnormal inflammatory disease model animal, the method comprising xenotransplanting into a non-human animal a graft derived from a patient suffering from an immune-abnormal inflammatory disease. In this case, the non-human animal preferably includes an immunodeficient mouse. Further, the graft to be xenotransplanted into a non-human animal preferably contains lymph node cells.

It is surprising that the model animal of an immune-abnormal inflammatory disease could be produced in this way. Without wishing to be bound by theory, this is because it has been assumed that in the case of an immune-abnormal inflammatory disease such as Castleman disease, when transplanted into an immunodeficient animal, immune-abnormal inflammation is induced and the animal cannot survive. Therefore, it should also be noted that the present disclosure could be provided by a model animal of an immune-abnormal inflammatory disease despite such expectations prior to the present disclosure.

### (Screening)

In one aspect of the present disclosure, the immune-abnormal inflammatory disease model animal of the present disclosure can be used to screen modulators of CXCL13, CXCR5, and/or peripheral helper T cells (Tph cells) [CXCL13/CXCR5/Tph]. In one embodiment, a small molecule medicine, a peptide medicine, an antibody medicine, a nucleic acid medicine (siRNA), or the like can be obtained by screening of the present disclosure. For example, in the screening of the present disclosure, small molecules that exhibit strong affinity for CXCL13 are screened in silico, or high-throughput screening are performed, or peptides that exhibit strong affinity for CXCL13 are screened in phage display. In one embodiment, affinity can confirm KD values with surface plasmon resonance (SPR) technology. The inhibitory activity of the interaction (IC50 value) can then be confirmed by SPR and further subjected to in vitro assays using Castleman disease-derived cell lines to evaluate the cell-based activity and toxicity of candidate small molecules or peptides.

After confirming sufficient activity and non-toxicity on a cell basis, in vivo effects can be confirmed by orally administering in the case of a small molecule or intravenously administering in the case of a peptide to a Castleman disease patient tissue transplanted model mouse using a highly immunodeficient mouse (NOD-SCID/IL2Rnull). The above process is preferably repeated until a highly active and highly selective small molecule or peptide is obtained, whereby a drug candidate molecule having a good absorption, distribution, metabolism, excretion and/or toxicity profile can be obtained.

The present disclosure has been described using various embodiments. The patents, patent applications, and documents cited herein for the description of the present disclosure are incorporated herein by reference in their entireties as if specifically set forth.

### Examples

Hereinafter, the present disclosure will be specifically described with reference to examples to facilitate understanding. However, the examples below are provided by way of example only, and are not provided for limiting the present disclosure. Accordingly, the scope of the present disclosure is not limited by the embodiments and the examples that are specifically described herein, and is limited only by the claims.

In the following examples, the patients and methods described below were used.

### (Characteristics of Patient)

From five control patients diagnosed with reactive lymphoma, follicular lymphoma or plasmablast lymphoma and seven patients who met criteria for iMCD, surgically resected swollen lymph nodes were obtained for diagnosis. The collected lymph node was cut into a size of 1 to 3 mm³ in RPMI 1640. Floating cells were collected from the medium. The cells were counted using Celltac Alpha MEK-6510K (NIHON KOHDEN CORPORATION) and subjected to subsequent analysis or cryopreserved as functional assay targets. All human studies were approved by the Institutional Review Board at Kyushu University Hospital.

### (Mice and Transplantation of Human LNCs)

Adult female mice of NSG (NOD.Cg-Prkdc^{scid}Il2rg^{tm1W1}/Sz) were used for xenotransplantation assay. The mice were bred and housed in a specific pathogen-free facility in microisolator cages at Kyushu University (Japan, FUKUOKA). Animal experiments were performed in accordance with institutional guidelines approved by the Animal Care and Use Committee of Kyushu University. For the reconstitution assay, selected cells (total 0.5 to 3×10⁶ cells per mouse) in RPMI 1640 were transplanted into 6 to 8 week old NSG mice that had been irradiated (2.2 Gy) intrathoracically via the tail vein as described in Y. Kikushige, et al. Cancer cell. 2011.

### (Antibodies and Cell Staining)

Eight weeks after inoculation with LNCs, spleen, liver, and bone marrow suspensions were prepared from the mice to assess engraftment of human cells. For FACS analysis, cells were stained with anti-human CD45 (HI30, biolegend), anti-mouse CD45 (30-F11, biolegend), and anti-Ter119 (TER-119, biolegend). The cells were further stained with anti-human CD3 (UCHT1, biolegend), anti-human CD4 (RPA-T4, biolegend), anti-human CD8a (RPA-T8, biolegend), anti-human CD19 (HIB19, biolegend), anti-human CD20 (2H7, biolegend), anti-human CD27 (M-T271, biolegend), anti-human CD34 (8G12, BD biosciences), anti-human CD35 (E11, biolegend), anti-human CD192 (CCR2) (K036C2, biolegend), and anti-human CD279 (PD-1) (EH12.2H7, biolegend). Non-viable cells were excluded by propidium iodide (PI) staining. After staining, the cells were analyzed using a BD FACS Aria II (BD Biosciences), BD FACS Aria IIIu (BD Biosciences), or Attune NxT Flow Cytometer (Thermo Fisher Scientific).

### (Immunohistochemical Staining)

Mouse spleens and livers were fixed in 10% neutral-buffered formalin and embedded in paraffin. Sections of 10 μm were deparaffinized and antigens were retrieved using citrate-based buffer (pH 6) for 10 min (H3300, Vector Laboratories). Slides were incubated in a blocking solution (3% bovine serum albumin and tris buffered saline with Tween 20) for 1 hour and incubated overnight with mouse anti-human CD3 (Dako, Clone F7.2.38, #M725401-2) and mouse anti-human CD20cy (Dako, Clone L26, #IR604). Slides were then incubated with an HRP-conjugated secondary antibody (Dako EnVision+ Dual Link System-HRP, Dako, #K4063). The slides were developed with diaminobenzidine (ImmPACT DAB Peroxidase (HRP) Substrate, Vector Laboratories, #SK-4105).

### (Imaging Mass Cytometry (IMC))

Tissue sections of 10 μm taken from the livers and spleens of iMCD-NSG mice were processed and stained with 11 markers for IMCD. The IMC antibodies are shown in Table 1.

### [Table 1]

Regions of interest detected by HE staining on the slides of the livers and spleens were acquired using Hyperion Imaging System (Fluidigm). IMC imaging mcd. files were exported into tiff files using R software.

### (Measurement of Cytokine/Chemokine and Immunoglobulin Protein)

The mouse sera obtained by xenotransplantation were assayed for 27 cytokines using the Bio-Plex Suspension Array System with Bio-Prex Pro Human Cytokine Screening 27-Plex Panel (Bio-Rad Laboratories Inc., CA, USA). The assay was performed for the following cytokines: IL-1β, IL-1Ra, IL-2, IL-4, IL-5, IL-6, IL-7, IL-9, IL-10, IL-12p70, IL-13, IL-15, IL-17, Eotaxin, FGF basic, GM-CSF, IFN-γ, IP-10, MIP-1a, PDGF-BB, MIP-1b, RANTES, TNF-α. CXCL13 was measured using a commercially available specific ELISA kit (ab269370; Abcam) according to the manufacturer's instructions. The values of each cytokine were used to generate heat maps.

Human IgG, IgA, and IgE in the mouse sera were measured according to the manufacturer's instruction using a commercially available specific ELISA kit (ab195215, ab196263, ab195216, abcam).

### (In Vivo Pharmacological Study)

Six to eight week old NSG mice (female) were irradiated with 2.2 Gy microwave radiation. The NSG mice were transplanted with LNCs of iMCD from the tail vein. The next day, a CXCL13-neutralizing antibody was intraperitoneally administered weekly a total of 4 times (15 μg/mouse, Biolegend, cat #A15151D), and rapamycin was intraperitoneally administered daily (20 μg/mouse, KOM, AG-CN2-0025-M005). An IgG antibody as a control was intraperitoneally injected weekly a total of 4 times.

### (Statistical Analysis)

Statistical analysis was performed using R (4.0.3). Applied statistical tests are shown in the legends or corresponding method sections. When P 0.05, it was regarded as a statistically significant difference.

### Example 1: Expression of Cachexia-Like Systemic Inflammation In Vivo by LN Cell Transplantation of Patients with iMCD-NOS into NSG Mice

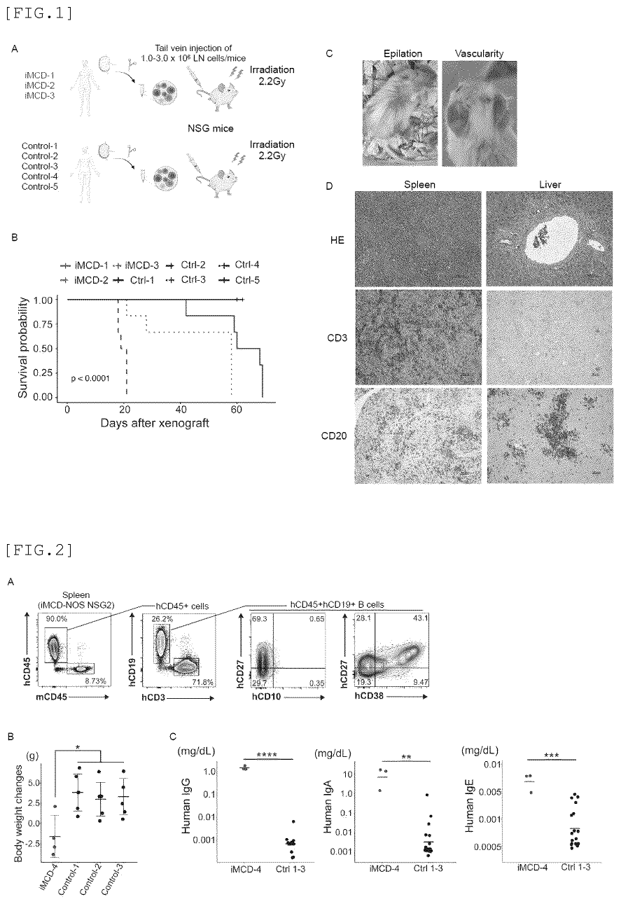

First, lymph node (LN) cells from a total of five independent control group patients (four patients with reactive lymphadenopathy and one patient with lymphoma) and three independent patients with iMCD-NOS (iMCD-NOS 1 to 3) were transplanted into NOD.Cg-Prkdc^{scid}Il2rg^{tm1Wj1}/Sz (NSG) mice irradiated with 2.2 Gy via tail vein injection (FIG. 1A). The clinical characteristics of the patients with iMCD-NOS and the control group are shown in Table 2.

### [Table 2]

Surprisingly, the LN cells from patients with iMCD-NOS caused fatal systemic inflammation in the recipient mice, and all the mice died within two months, whereas none of the NSG mice transplanted with reactive LN cells (control NSG mice) died in all the cases examined (FIG. 1B). Interestingly, the recipient mice transplanted with LN cells from patients with iMCD-NOS (iMCD-NOS NSG mice) showed body weight loss, depilation of the whole body and vasodilation in the ear, known as the clinical conditions of iMCD (FIG. 1C). All iMCD-NOS NSG mice died due to cachexia by week 10. On the other hand, all control NSG mice survived to week 10.

Hematoxylin and eosin staining revealed diffuse infiltration of human lymphocytes in the spleens of iMCD-NOS NSG mice (FIG. 1D, upper row). Immunohistochemical staining confirmed diffuse infiltration of human CD3⁺ T cells (FIG. 1D, middle row) and CD20⁺ B cells (FIG. 1D, lower row) in the spleens and livers of iMCD-NOS NSG mice. Combining these results, it can be seen that xenotransplantation of LN cells from patients with iMCD-NOS causes fatal systemic inflammation, reproducing iMCD-NOS-like conditions in mouse organisms.

### Example 2: Infiltration of Mature B Cells and Elevation of Plasma Levels of IgG, IgM, and IgE in iMCD-NSG Mice

FACS analysis revealed that human (h) CD45⁺ cells including hCD3⁺ T cells and hCD19⁺ B cells proliferated in the spleens of iMCD-NOS NSG mice (left in FIG. 2A).

Engraftment and infiltration of not only CD8⁺ T cells but also CD4⁺ T cells were observed in the bone marrow, spleens and livers of iMCD-NOS NSG mice. Consistent with the development of a condition like iMCD-NOS in mouse organisms, hCD10⁻hCD19⁺hCD20⁺hCD27⁺hCD38⁺ plasmablasts were expressed in the spleens of recipient mice (FIG. 2A, right). Importantly, rearrangement analysis of TCR/IGH genes by PCR revealed that these expressed human CD3⁺ T cells and CD19⁺ B cells were polyclonal in lymph node cells of all patients examined (not shown).

Since memory cells to plasmablast B cells were detected in iMCD-NOS NSG mice, it was examined whether plasma cells proliferated in MCD-NOS NSG mice secrete human gamma-globulin in the sera of recipient mice. For this purpose, LN cells from other patients with iMCD-NOS (iMCD-NOS 4) were xenotransplanted into NSG mice, and iMCD-like findings were reproduced in the mice. Consistent with systemic inflammation, iMCD-NOS NSG (iMCD-NOS 4) showed significant body weight loss after 8 weeks of xenotransplantation (FIG. 2B). Serum levels of human IgG, IgA and IgE of iMCD-NOS NSG (iMCD-NOS 4) were significantly higher than those of control NSG mice (FIG. 2C).

### Example 3: T Cells are Required for iMCD-NSG Mouse Engraftment, and Hypergammaglobulinemia, and Cytokine Storm Reproduction

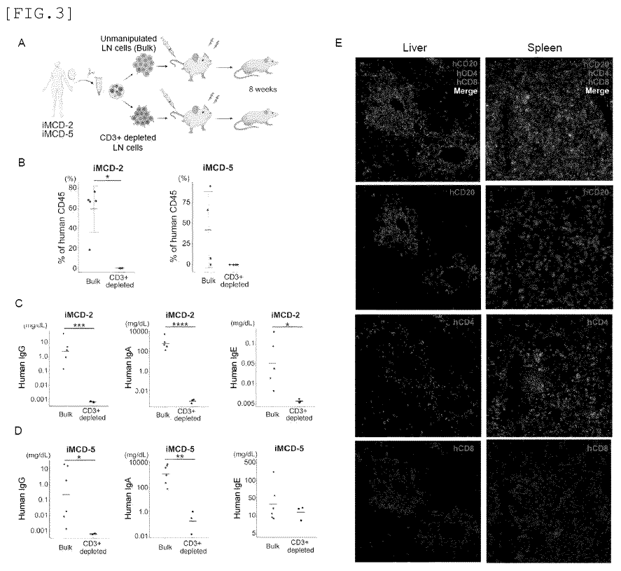

To examine which lineages of cells are involved in the development of iMCD-NOS-like systemic inflammation in mice, the same number of unmanipulated LN cells and T cell-depleted LN cells from two independent patients with iMCD-NOS (iMCD-NOS 2 and 5) were xenotransplanted (FIG. 3A). Xenotransplantation of unmanipulated LN cells from patients with iMCD-NOS showed an increase in human cells. Transplantation of T cell-depleted LN cells from identical patients did not show human cells in the spleen. (FIG. 3B). Furthermore, significantly higher human γ-globulin production was observed in NSG mice transplanted with unmanipulated LN cells in both cases (FIG. 3C for iMCD-NOS 2 and FIG. 3D for iMCD-NOS 5, respectively). These results strongly suggest that the interaction between human B cells and T cells plays a critical role in the development of iMCD-like systemic inflammation and expansion of plasmablasts in iMCD-NOS NSG mice. Consistent with the critical role of T-B interaction in vivo, co-localization of hCD4⁺ T cells, hCD8⁺ T cells, and hCD20⁺ B cells in the spleens and livers of iMCD-NOS NSG mice was confirmed using Hyperion multicolor imaging system (FIG. 3E).

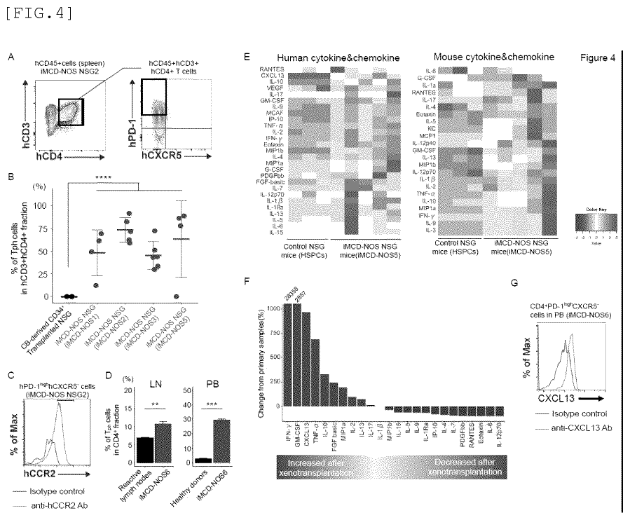

Since human T cells present in iMCD-NOS LN were found to play a pivotal role in the development of iMCD-like disease through the interaction with B cells in the recipient mice, human T cell subsets in the mice were analyzed. Interestingly, hCD3⁺hCD4⁺hPD-1^{high}hCXCR5⁻ Tph cells infiltrated the spleens of iMCD-NOS NSG mice (FIG. 4A), whereas no infiltration was seen in humanized NSG mice that reconstituted human normal hematopoiesis by xenotransplantation of cord blood (CB)-derived CD34+ hematopoietic stem/progenitor cells (HSPCs) (FIG. 4B). Importantly, infiltration of human Tph cells was observed in NSG mice transplanted with total unmanipulated LN cells from four independent patients with iMCD-NOS (iMCD-NOS1, iMCD-NOS2, iMCD-NOS3, and iMCD-NOS5) (FIG. 4B). Importantly, the infiltrated hCD3⁺hCD4⁺hPD-1^{high}hCXCR5⁻ cells expressed hCCR2, an important marker for Tph cells (FIG. 4C). Furthermore, infiltration of PD-1^{high}hCXCR5⁻ Tph cells was confirmed in LN and peripheral blood (PB) of a patient with iMCD-NOS (iMCD-NOS 6) (FIG. 4D). It has been shown that Tph cells promote maturation and proliferation of B cells at a local site via the production of several cytokines and chemokines. Therefore, Tph cells are considered to play a critical role in the development of iMCD-like systemic inflammation through the production of several cytokines and chemokines.

The next aim was to determine which cytokines and chemokines are involved in the pathogenesis of iMCD. Therefore, human and mouse cytokines and chemokines in the sera of iMCD-NOS NSG mice (iMCD-NOS5) (n=5) and NSG mice transplanted with CB-derived CD34⁺ HSPC and reconstituted with human normal hematopoiesis (n=3) were quantified. iMCD-NOS NSG mice showed significant elevation of multiple human cytokines and chemokines in the sera, whereas NSG mice transplanted with normal human cord blood did not (FIG. 4E, left). Importantly, mouse cytokines and chemokines were also significantly elevated in iMCD-NOS NSG mice, reflecting severe systemic inflammation caused by xenotransplanted LN cells from patients with iMCD-NOS (FIG. 4E, right).

Among the elevated human cytokines from iMCD-NOS NSG mice, it is aimed to identify cytokines and chemokines that could cause systemic inflammation in iMCD-NOD NSG mice of the present disclosure. For this purpose, changes in cytokines and chemokines were evaluated by comparing serum concentrations in iMCD-NOS NSG mice and the original patients. FIG. 4F summarizes changes in serum concentrations of cytokines and chemokines in humans.

Human cytokines/chemokines that were elevated in iMCD-NOS NSG mice were shown on the left side in the figure, and reduced cytokines/chemokines were shown on the right side in the figure. IFN-γ, GM-CSF, CXCL13, TNF-α were found to be dramatically elevated compared to those in the sera of the original patients (iMCD-NOS5). It has been reported that all of these cytokines and chemokines are expressed in Tph cells. Furthermore, a recent study revealed that CXCL13 is the most elevated chemokine during disease exacerbation in patients with iMCD, confirming expression of CXCL13 in purified Tph cells of the patient (iMCD-NOS6) (FIG. 4G). Based on these results, it was presumed that CXCL13 derived from Tph cells plays a crucial role in the development of iMCD-like systemic inflammation in vivo.

### Example 4: Suppression of Early Death in iMCD-NOS NSG Mice by Human CXCL13 Neutralizing Antibody

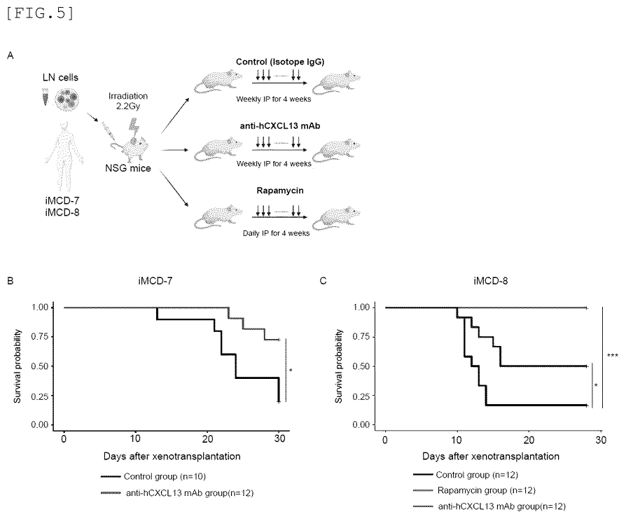

To verify whether CXCL13 is involved in the pathogenesis of iMCD-NOS, therapeutic experiments were performed using the PDX model of the present disclosure. LN cells from two independent patients with iMCD-NOS (iMCD-NOS7 and iMCD-NOS8) were transplanted into NSG mice. FIG. 5A shows an experimental model. Twenty-four hours after xenotransplantation, recipient mice were randomly assigned to a control group and a treatment group. Control mice were intraperitoneally injected with mouse isotype IgG antibody (15 ug/mouse) once a week for 4 weeks. NSG mice in the treatment group were intraperitoneally injected with rapamycin (40 ug/mouse) daily for 4 weeks, or intraperitoneally injected with an anti-hCXCL13 mouse IgG1 antibody (15 ug/mouse), which could neutralize chemotactic activity of human CXCL13, once a week for 4 weeks.

First, therapeutic experiments were performed using iMCD-NOS LN cells (iMCD-NOS7), and it was found that the survival of iMCD-NOS NSG mice was significantly improved by administration of anti-hCXCL13 antibody as compared to that of the control subjects (FIG. 5B). FIG. 5C is a survival analysis of iMCD-NOS NSG mice (iMCD-NOS8) treated with isotype IgG, rapamycin, and anti-hCXCL13 antibody. The control mice developed fatal inflammation, and most of them died within 20 days. Rapamycin treatment significantly improved the survival of iMCD-NOS NSG mice, and about half of the mice were able to survive within the observation period. Furthermore, anti-hCXCL13 antibody administration completely blocked the development of fatal inflammation and none of the iMCD-NOS NSG mice died.

These results suggest that hCXCL13, presumably secreted from Tph cells, plays a pivotal role in promoting systemic inflammation reproduced in the iMCD-NOS NSG mice of the present disclosure through interaction with human B cells.

### Example 5: Therapeutic Effect on iMCD-NOS NSG Mice by CXCL13 Neutralizing Antibody

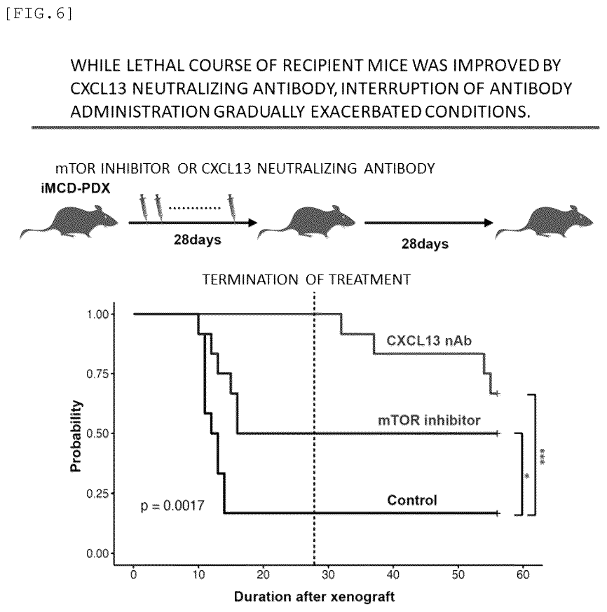

In order to show the long-term therapeutic effect of CXCL13 on iMCD, iMCD-NOS NSG mice were administered a CXCL13 neutralizing antibody and mTORC1 inhibitor (rapamycin), a conventional therapy for iMCD (FIG. 6). The graph shown in the lower part of FIG. 6 is a survival curve when a CXCL13 neutralizing antibody was administered or when an mTORC1 inhibitor was administered. On Day 28 of treatment, the mouse cohort administered the CXCL13 neutralizing antibody had significantly improved overall survival compared to the mouse cohort administered the mTORC1 inhibitor and the mouse cohort administered PBS as a control. In addition, when observation was further performed for 28 days after termination of the treatment, it was found that in the mouse cohort treated with the CXCL13 neutralizing antibody, treatment interruption exacerbates conditions, leading to cachexia and death.

### Notes

As described above, the preferred embodiments of the present disclosure have been used as examples of the present disclosure, but it is understood that the scope of the present disclosure should be construed only by the claims. It is understood that the patents, patent applications, and other documents cited herein should be incorporated herein by reference in their entireties as if specifically set forth herein.

### INDUSTRIAL APPLICABILITY

The present disclosure provides novel compositions for treating idiopathic multicentric Castleman disease, a refractory lymphoproliferative disease, which are available in the pharmaceutical industry.

1. A composition for preventing or treating an immune-abnormal inflammatory disease, comprising a modulator of CXCL13, CXCR5, and/or peripheral helper T cells (Tph cells) [CXCL13/CXCR5/Tph].

2. The composition according to claim 1, wherein the modulator is selected from the group consisting of proteins, polypeptides, oligopeptides, peptides, polynucleotides, oligonucleotides, nucleotides, nucleic acids (including, for example, cDNA, DNA such as genomic DNA, and RNA such as mRNA), polysaccharides, oligosaccharides, lipids, organic small molecules (e.g., hormones, ligands, information transmitters, organic small molecules, molecules synthesized by combinatorial chemistry, small molecules that can be utilized as pharmaceuticals (e.g., small molecule ligands and the like), and the like), and complex molecules thereof.

3. The composition according to claim 1, wherein the modulator is:
an anti-CXCL13 antibody, a nucleic acid medicine for CXCL13, or a CXCL13 inhibitor or peptide,
an anti-CXCR5 antibody, a nucleic acid medicine for CXCR5, or a CXCR5 inhibitor or peptide, or
an anti-Tph cell antibody, a nucleic acid medicine for Tph cell, or a Tph cell inhibitor or peptide.

4. The composition according to claim 1, wherein the modulator is a modulator of CXCL13-CXCR5 interaction.

5. The composition according to claim 1, wherein the modulator is a modulator of Tph cells.

6. The composition according to claim 3, wherein the anti-CXCL13 antibody is a neutralizing antibody against CXCL13.

7. The composition according to claim 3, wherein the anti-CXCL13 antibody is a chimeric antibody, humanized antibody, or human antibody against CXCL13.

8. The composition according to claim 1, wherein the disease is a CXCL13-mediated disease.

9. The composition according to claim 1, wherein the disease comprises Castleman disease, rheumatoid arthritis, plasmacytosis, hyperimmunoglobulinemia, anemia, nephritis, cachexia, multiple myeloma, mesangial proliferative nephritis, systemic lupus erythematosus, systemic scleroderma, Crohn's disease, ulcerative colitis, pancreatitis, psoriasis, juvenile chronic arthritis, or systemic juvenile idiopathic arthritis, vasculitis, Kawasaki disease, TAFRO syndrome, and Poems syndrome.

10. The composition according to claim 1, wherein the disease comprises idiopathic multicentric Castleman disease.

11. An immune-abnormal inflammatory disease model animal.

12. The model animal according to claim 11, wherein the immune-abnormal inflammatory disease is Castleman disease.

13. The model animal according to claim 11, wherein the immune-abnormal inflammatory disease is idiopathic multicentric Castleman disease.

14. The model animal according to claim 11, wherein the animal is an immunodeficient animal, and the immunodeficient animal contains lymph node cells derived from other than the immunodeficient animal.

15. The model animal according to claim 14, wherein the lymph node cells are lymph node cells of a patient with iMCD-NOS.

16. A method for producing an immune-abnormal inflammatory disease model animal, the method comprising a step of xenotransplanting into a non-human animal a graft derived from a patient suffering from an immune-abnormal inflammatory disease.

17. The method according to claim 16, wherein the non-human animal comprises an immunodeficient animal.

18. The method according to claim 16, wherein the non-human animal comprises an immunodeficient mouse.

19. The method according to claim 16, wherein the graft comprises lymph node cells.

20. The method according to claim 19, wherein the lymph node cells are lymph node cells of a patient with iMCD-NOS.

21. The method according to claim 16, wherein the disease comprises Castleman disease, rheumatoid arthritis, plasmacytosis, hyperimmunoglobulinemia, anemia, nephritis, cachexia, multiple myeloma, mesangial proliferative nephritis, systemic lupus erythematosus, systemic scleroderma, Crohn's disease, ulcerative colitis, pancreatitis, psoriasis, juvenile chronic arthritis, or systemic juvenile idiopathic arthritis, vasculitis, Kawasaki disease, TAFRO syndrome, and Poems syndrome.

22. The method according to claim 16, wherein the disease comprises idiopathic multicentric Castleman disease.
